(19) United States
(12) Patent Application Publication
(10) Pub. No.: US 2005/0251148 A1
(43) Pub. Date: Nov. 10, 2005

(54) Method and device for determining the position of a knee-joint endoprosthesis

(75) Inventors: Dirk Friedrich, Tuttlingen (DE); Francois Leitner, Uriage (FR)

Correspondence Address: Lipsitz & McAllister, LLC, 755 MAIN STREET, MONROE, CT, 06468, US

(73) Assignee: AESCULAP AG & Co. KG, Tuttlingen (DE)

(21) Appl. No.: 11/116,104

(22) Filed: Apr. 26, 2005

Related U.S. Application Data
(63) Continuation of application No. PCT/EP02/12319, filed on Nov. 5, 2002.

(51) Int. Cl. A61B 17/58

(52) U.S. Cl. 606/88

(57) ABSTRACT

To be able to determine the insertion position of the parts of an artificial knee joint without changing the original knee joint, a method is proposed for determining the position of the tibial part and/or the femoral part of a knee-joint endoprosthesis in relation to the proximal tibial head or to the distal femur in which the position of the femur and of the tibia are monitored by means of a navigation system, in which the distal femur and the proximal tibial head are laterally and medially displaced with a defined force into a spread position by means of a distraction appliance when the knee is straightened and bent, and the relative positions of the femur and the tibia, and consequently the size of the gap between the femur and the tibia, are thereby respectively determined, in which various virtual relative positions of the femur and the tibia are calculated according to geometrical data of the knee-joint endoprosthesis and to different assumed positions of the tibial part on the tibia and/or of the femoral part on the femur when the knee is straightened and bent, and in which an assumed position in which the virtual relative position of the femur and the tibia when the knee is straightened and bent differs from the spread position in a specified manner is determined as a selected position.

This application is a continuation of international application No. PCT/EP02/12319 filed on Nov. 5, 2002.

The present disclosure relates to the subject matter disclosed in international application No. PCT/EP02/12319 of Nov. 5, 2002, which is incorporated herein by reference in its entirety and for all purposes.

### BACKGROUND OF THE INVENTION

The invention relates to a method for determining the position of the tibial part and/or the femoral part of a knee-joint endoprosthesis in relation to the proximal tibial head or to the distal femur. Furthermore, the invention relates to a device for carrying out this method.

In the implantation of a knee-joint endoprosthesis, endoprostheses which comprise two or three parts, for example a tibial part which can be fixed to the tibia, a femoral part which can be fixed to the femur, and an intermediate part, which is disposed between the femoral part and the tibial part, are often used. The tibial part and the femoral part must be respectively connected to the tibia and the femur in such a way that the kinematics of the original knee joint are reproduced as well as possible. To achieve this, the tibial head and the femur must be appropriately recessed by sawn cuts, so that the tibial part and the femoral part engage with the tibia and femur in the desired position; this position of engagement is responsible for the optimum kinematics of the knee joint.

It is known for the implantation of a knee joint to monitor the original kinematics of the knee and also the kinematics of the knee after the implantation of the endoprosthesis by monitoring the position both of the femur and of the tibia by a navigation system known per se. Such a navigation system can continuously determine the spatial position of specific marking elements, the marking elements being, for example, elements with a number of radiating transmitters, for instance infrared diodes, in other cases marking elements with a number of spaced-apart reflectors, which for example reflect infrared radiation well. The navigation system records the radiation emanating from the marking element and can in this way determine the spatial position of the marking element, and consequently also the position of the body part to which the marking element is fixed. Navigation systems of this type are regularly used in knee operations to monitor the sequence of movements of the femur and the tibia and also to record anatomical data of the femur and of the tibial head by means of suitable engaging or contacting instruments (DE 100 31 887 A1).

For a knee operation to be successful, it is important that, after the insertion of the implant, the femur and the tibia are uniformly tensioned with respect to one another on the lateral and medial sides by ligaments connecting the femur and the tibia, to be precise as far as possible both in the straightened position and in the bent position of the leg. To achieve this, it is known to insert trial implants and to measure the tensions obtained for these trial implants once they have been inserted, for example with the aid of a distraction appliance, which measures the forces holding the femur and the tibia together by spreading the gap between them open.

This method is extremely laborious and entails the risk that, because of measuring errors, the desired tensioning conditions are not achieved after all once the operation has been carried out; this can have the effect that the surfaces of the joint are tensioned too little, or else too much, with respect to one another after the operation.

It is an object of the invention to devise a method of the generic type in such a way that it can be determined during the course of the operation, and without making any changes to the original knee joint, how the parts of the endoprosthesis are to be implanted to reproduce the natural tensioning conditions in the knee joint in the desired way, or if appropriate to modify them.

### SUMMARY OF THE INVENTION

This object is achieved according to the invention by a method for determining the position of the tibial part and/or the femoral part of a knee-joint endoprosthesis in relation to the proximal tibial head or to the distal femur in which the position of the femur and of the tibia are monitored by means of a navigation system, in which the distal femur and the proximal tibial head are laterally and medially displaced with a defined force into a spread position by means of a distraction appliance when the knee is straightened and bent, and the relative positions of the femur and the tibia, and consequently the size of the gap between the femur and the tibia, are thereby respectively determined, in which various virtual relative positions of the femur and the tibia are calculated according to geometrical data of the knee-joint endoprosthesis and to different assumed positions of the tibial part on the tibia and/or of the femoral part on the femur when the knee is straightened and bent, and in which an assumed position in which the virtual relative position of the femur and the tibia when the knee is straightened and bent differs from the spread position in a specified manner is determined as a selected position.

The method described can therefore be carried out without having to make changes to the tibia or femur. It is sufficient to expose the knee joint and then, by spreading open the gap between the tibial head and the femur when the knee is straightened and when it is bent, to determine the respective relative position of the femur and the tibia, and consequently the width of the gap between the tibial head and the femur. In a next step, positions intended for the selected parts of the prosthesis on the tibia and on the femur are established. This only takes place virtually, that is to say not in actuality, in that engagement surfaces on which the parts of the prosthesis can be placed against the femur and the tibia, for example sawing planes, are determined.

With the engagement surfaces assumed in this way, and consequently the positions of the parts of the implant on the femur and/or the tibia assumed in this way, it can then be calculated how the femur and the tibia move in relation to one another in the bending movement, if the geometrical data of the parts of the implant are taken as a basis for this calculation. These geometrical data specify how the parts of the implant move in relation to one another, and the virtual relative positions of the femur and the tibia during the bending movement can be calculated from the assumed positions of the parts of the implant on the femur and the tibia on the one hand and the relative movement of the implants to one another on the other hand. These positional data are virtual because no part of the prosthesis is moved in actuality, but instead this movement is performed purely by calculation.

The virtual positional data for a specific assumed position of the parts of the prosthesis are subsequently compared with the relative positions which have been recorded on the anatomical knee joint during the spreading-open with the aid of the distraction appliance.

Optimum reproduction of the body's own kinematics can be achieved if, according to a preferred embodiment, a selected position is determined in such a way that the virtual relative position of the femur and the tibia coincides with the spread position. A sequence of movements which corresponds to the natural sequence of movements is then obtained, the tensioning of the system of ligaments in the straightened and bent positions being identical to the natural state.

It is, however, of course also possible for the surgeon specifically to desire that the virtual relative position differs from the actual relative position, for example for the correction of a malposition. There is then the possibility of continuing to calculate virtual relative positions by variation of the assumed position of the parts of the implant until the virtual relative position which corresponds to the desired difference from the actual relative position is found, in other words the movement of the knee is in this way simulated according to differently assumed positions of the parts of the prosthesis, without working of the tibial head or the femur being necessary for this.

For example, it may be provided that a selected position is determined in such a way that the size of the gap laterally and medially between the femur and the tibia in the bent position and/or the straightened position of the knee is at least approximately equal.

The position of the parts of the prosthesis can be differently assumed in a variety of ways, for example in the case of a first preferred embodiment it may be provided that, for the calculation of various virtual relative positions, the assumed position of the femoral part is displaced by displacement of the femoral part perpendicularly to the longitudinal axis of the femur while remaining parallel to itself.

The longitudinal axis of the femur may in this case be identical to the mechanical axis of the femur which connects the knee joint to the hip joint. This different relative position corresponds to a different thickness of the recessed joint surface at the distal end of the femur.

In the case of a further preferred embodiment it is provided that, for the calculation of various virtual relative positions, the assumed position of the femoral part is displaced by its displacement in the anterior-posterior direction while remaining parallel to itself. With such a displacement, a change in the distance of the dorsal joint surface of the femoral part is obtained, so that this joint surface is at various distances from the tibial head when the knee is bent.

In the case of a further preferred variation of the assumed position of the femoral part it is provided that, for the calculation of various virtual relative positions, the assumed position of the femoral part is changed by its pivoting about an anterior-posterior extending axis. Such a pivoting leads to a correction of the varus-valgus position of the knee, i.e. as a result the width of the gap between the femur and the tibial part is changed laterally and medially even when the knee is straightened.

A further possibility for changing the assumed position envisages that, for the calculation of various virtual relative positions, the assumed position of the femoral part is changed by its pivoting about a medial-lateral extending axis. This leads to an inclination of the joint surfaces with respect to the longitudinal axis of the femur and may be of significance for certain corrections.

The said variations may be performed individually or in combination, and it is clear that each of these changes to the position of the femoral part leads to a changed kinematic behavior of the knee joint. The described simulation of this movement obtained by the calculation of the respectively corresponding virtual relative positions of the femur and the tibia allow the surgeon to establish the consequences of a change to the position in each case by comparison of the virtual relative positions with the spread position in the straightened and bent knee and to keep varying the assumed position until on the one hand the desired coincidence with the spread position is achieved as well as possible and on the other hand a possibly desired correction, for example a varus-valgus correction, is achieved.

In addition to differently assumed positions, it is also possible according to a development of the invention for differently dimensioned tibial parts and/or femoral parts to be taken as a basis for the calculation of various virtual relative positions. It is therefore possible to assume tibial or femoral parts of different sizes in this simulation and to calculate their effects on the virtual relative positions in the simulation. Consequently, with the choice of the dimensions of the tibial or femoral parts on the one hand and the variation of the position of these parts on the other hand, the surgeon has the choice of many possible ways of influencing the kinematics of the knee, and the result of these different parameters can be examined in advance during the operation by the simulation described, without the tibia or femur already having to be worked on.

It is advantageous if, in the determination of the spread position, the gap between the femur and the tibial head is widened to the maximum extent. The system of ligaments becomes increasingly stiffer during the widening and, once extended by a certain amount, is virtually unable to extend any further, i.e. the distraction reaches saturation. The achievable extension in this range is relatively independent of the extending force expended, and for this reason it is advantageous to distract into this range; this produces results which can be reproduced well.

During the bending movement of the knee and also when the distraction appliance is engaged, the actual gap between the sliding surfaces of the femur and the tibial head can only be accurately determined from the monitoring of the position of the femur and the tibia if geometrical data on the shape of the femur and the tibia are additionally available. It is therefore envisaged according to a further development of the invention that, to determine the size of the gap between the femur and the tibia, the contour of the proximal tibial head and the contour of the distal femur are determined by the engagement of at least one navigated engaging element on these contours. This is a technique known per se for contour determination; the engagement of a navigated engaging element allows the navigation system to localize accurately the point of engagement or the line of engagement by the respective position of the engaging element in relation to the femur and the tibia, and from this to calculate geometrical data for the overall contour of the tibial head and of the femur.

For example, it may be provided that an engaging element has a contacting tip, with which various points of the contours are sensed.

In the case of another configuration it is provided that an engaging element has a planar engagement surface, which is placed against the contour to be determined.

In the case of a particularly preferred embodiment it is provided that an engaging element has two mutually perpendicular engagement surfaces, which are jointly placed against the contour to be determined. This is advantageous for example to determine the contour of the condylar surfaces of the femur; the two engagement surfaces can then be placed against the distal condylar surface or the dorsal condylar surface, so that information on the contours in this region can be obtained.

The comparison of the spread position and the virtual relative position respectively calculated by the simulation will preferably take place with the aid of a visual display unit or a display on which information derived from these data is displayed to the surgeon; for example, according to a preferred embodiment it may be provided that the size of the medial and lateral gaps in the straightened and bent positions of the knee in the respectively calculated virtual relative positions are shown on a display. The surgeon can now for example change the assumed positions in such a way that the size of the gap becomes the same medially and laterally and as far as possible in the stretched and bent states.

Furthermore, it is advantageous if the position of the femoral part assumed for the determination of various virtual relative positions is displayed on a display in relation to geometrical data of the femur, for example an image of the femur corresponding to the geometrical data. The surgeon can then for example read off directly the inclination of an engagement surface of the femoral part in relation to the longitudinal axis of the femur or a similar measure, and perform a corresponding variation of the assumed position in such a way that this read-off parameter corresponds to what the surgeon envisages.

While it is readily possible to carry out the method described without the tibial head and the femur having been worked on, in certain cases it may be advantageous if, before spreading open the gap between the femur and the tibial head, the proximal joint surface of the tibial head is recessed along a plane which is perpendicular to the longitudinal axis of the tibia. As a result, an engagement surface for the distraction appliance is obtained, and this engagement surface can also be used as an engagement surface for the tibial part, so that the adaptation of the endoprosthesis then takes place exclusively in the femoral part.

The invention is also based on the object of providing a system with which the position of the parts of a knee-joint endoprosthesis can be determined.

This object is achieved according to the invention by a system for determining the position of the tibial part and/or the femoral part of a knee-joint endoprosthesis in relation to the proximal tibial head or to the distal femur with a navigation system for monitoring the position of the femur and the tibia by means of marking elements which can be fixed on the femur and tibia, with a distraction appliance, which displaces the distal femur and the proximal tibial head laterally and medially with a defined force into a spread position when the knee is straightened and bent, with a data processing system, which determines the relative position of the femur and the tibia during the distraction, and consequently the size of the gap between the femur and the tibia, and calculates various virtual relative positions of the femur and the tibia according to geometrical data of the knee-joint endoprosthesis and different assumed positions of the tibial part on the tibia and/or of the femoral part on the femur when the knee is straightened and bent.

In this case it may be provided that a display which displays data corresponding to the relative position of the femur and the tibia during the distraction and data corresponding to the virtual relative positions for the purpose of their comparison is associated with the data processing system. In particular, it may be provided in this case that the data processing system shows on the display the size of the medial and lateral gaps in the straightened and bent positions of the knee in the respectively calculated virtual relative positions.

It is also advantageous if the data processing system displays on the display the position of the femoral part assumed for the determination of various virtual relative positions in relation to geometrical data of the femur.

According to a preferred embodiment, a device of this type comprises at least one navigated engaging element, which can be placed against the contour of the proximal tibial head and the contour of the distal femur to determine the size of the gap between the femur and the tibia.

It may be provided in this case that an engaging element has a contact tip, with which various points of the contours can be sensed.

In the case of another embodiment it is provided that an engaging element has a planar engagement surface, which is placed against the contour to be determined.

The engaging element may in this case have two mutually perpendicular engagement surfaces, which are jointly placed against the contour to be determined.

The following description of preferred embodiments of the invention serves for a more detailed explanation in conjunction with the drawing.

### BRIEF DESCRIPTION OF THE DRAWINGS

FIG. 1 shows a schematic overall view of a system for determining the position of parts of a prosthesis in the knee of a patient;

FIG. 2 shows a schematic front view of a bent knee with a navigated femur, a navigated tibia and a navigated engaging plate on a recessed tibial surface;

FIG. 3 shows a lateral view of a navigated femur with a navigated engaging element with two mutually perpendicular engagement surfaces and with a navigated engaging element in the form of a contact tip;

FIG. 4 shows a view similar to FIG. 2 with a distraction appliance inserted between the femur and the tibial head;

FIG. 5 shows a side view of the bent knee of FIG. 4;

FIG. 6 shows a view similar to FIG. 4 in the case of a knee joint in the distracted position, without the distraction appliance being represented;

FIG. 7 shows a view similar to FIG. 6 in the case of a straightened knee;

FIG. 8 shows a display for the medial and lateral gap widths when the knee is bent and straightened;

FIG. 9 shows a front view of a femur with assumed positions of the femoral part of an endoprosthesis displaced in the direction of the longitudinal axis of the femur;

FIG. 10 shows a view similar to FIG. 9 with an engagement surface pivoted about an anterior-posterior axis;

FIG. 11 shows a side view of a femur with an engagement surface pivoted about a medial-lateral axis;

FIG. 12 shows a view similar to FIG. 11 with an engagement surface displaced in the anterior-posterior direction;

FIG. 13 shows a schematic side view of a knee joint with an inserted endoprosthesis and

FIG. 14 shows a flow diagram to describe the sequence of the method when determining the position of the parts of the endoprosthesis in relation to the tibia and the femur.

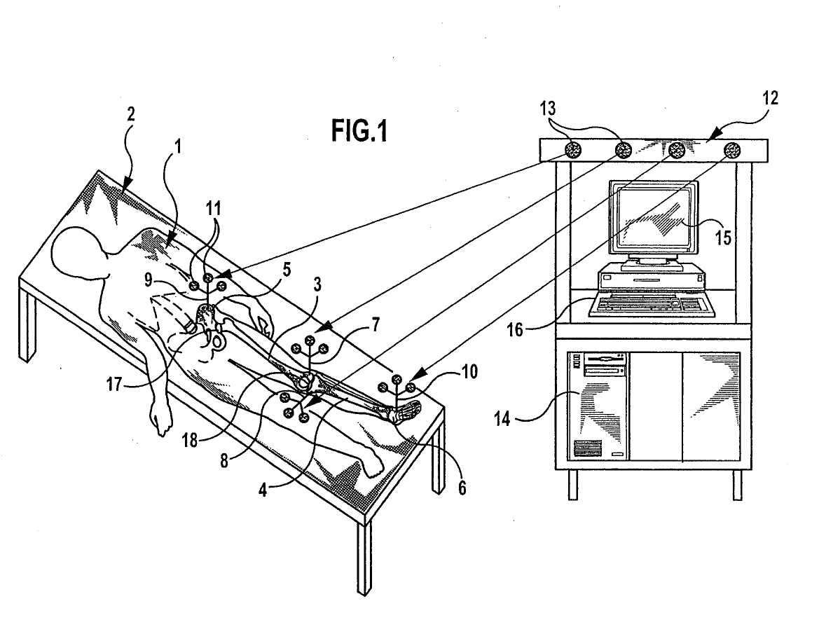

### DETAILED DESCRIPTION OF THE INVENTION

To replace a knee joint by a knee-joint endoprosthesis, the patient 1 is placed on an operating table 2, and the knee joint is opened up in a way known per se. Marking elements 7, 8, 9 and 10 are rigidly fixed at least to the femur 3 and to the tibia 4, and preferably also to the hip bone 5 and to the foot 6 of the patient, for example by screwing in a bone screw. Each of these marking elements carries three spaced-apart emitters 11, which may be active radiation transmitters for ultrasound radiation, infrared radiation or similar radiation, or else passive reflection elements for such a radiation, which then reflect, and thereby emit, radiation impinging on them. These marking elements operate together with a navigation system 12 with a number of radiation receivers 13, which establishes the spatial position and orientation of the marking elements and feeds data corresponding to this position to a data processing system 14. The data processing system 14 is equipped with a display 15 in the form of a screen and with a keyboard 16 for the input of additional data.

To prepare for implantation of a knee-joint endoprosthesis, firstly the mechanical axes of the femur 3 and of the tibia 4 are determined. This mechanical axis of the femur 3 is obtained, for example, from the line joining the hip joint 17 and the knee joint 18; these joints can be determined with the aid of the navigation system 12 in a way known per se, by the femur and the hip joint on the one hand and the femur and the tibia on the other hand being moved with respect to one another; in the same way, the mechanical axis of the tibia can be determined as the line joining the foot joint and the knee joint by relative movement of the femur and the tibia on the one hand and the tibia and the foot on the other hand. By this movement, the marking elements rigidly connected to the femur, the tibia, the hip bone and the foot are also moved on paths defined by the joints, and the data processing system 14 can then determine from these paths the position of the joints in relation to the marking elements, and consequently also in relation to the body parts to which the marking elements are fixedly connected.

In this way, a mechanical axis of the femur and a mechanical axis of the tibia which correspond approximately to the longitudinal direction of these bones are obtained.

In a first step of the operation, after determining the mechanical axes of the femur and the tibia, the tibial head 19 is prepared, that is to say the proximal end of the tibia. The joint surface facing the femur 20, that is to say the distal end of the femur 3, is removed by a planar saw cut; the resultant sawing plane 21 extends perpendicularly to the previously determined mechanical axis of the tibia and is generally located only a few millimeters beneath the proximal end of the tibial head 19.

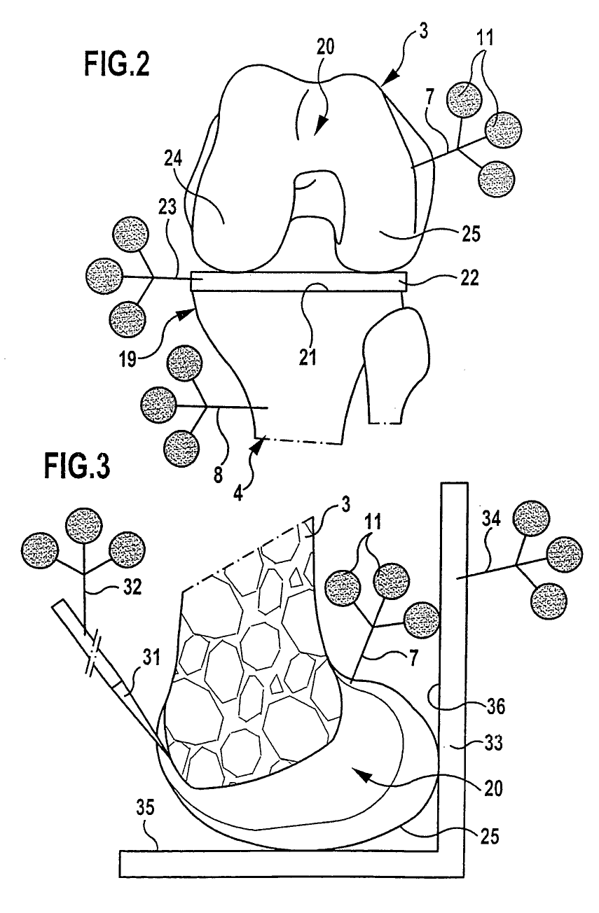

The precise position of the sawing plane 21 is checked once again by placing onto the sawing plane 21 a plate-shaped engaging element 22, which for its part is rigidly connected to a marking element 23, so that the navigation system can determine the spatial position of the engaging element 22, and with it also the position of the sawing plane 21 (FIG. 2).

In a next step, a distraction appliance 26 is pushed into the intermediate space between the sawing plane 21 of the tibial head 19 on the one hand and the mutually adjacent and spaced-apart joint surfaces 24, 25 of the femur 20 on the other hand. This distraction appliance has two spreading elements 27, 28, which can be displaced in relation to one another; a lower web-shaped spreading element 27 is placed against the sawing plane 21, an upper spreading element 28, extending parallel to said lower spreading element, is placed against one of the two joint surfaces 24, 25. Each of these joint surfaces 24, 25 has a distraction appliance 26 of its own placed against it, so that in each case one or the other of the joint surfaces 24 and 25 can be positioned at a spacing from the sawing plane 21 when the two spreading elements 27, 28 are moved apart.

The construction of a distraction appliance may vary greatly; there are a large number of distraction appliances of this type; all that is important is that, by displacing the spreading elements 27, 28 with respect to one another, for example by means of spreading instruments 29 acting on them (only represented very schematically in FIG. 5), the intermediate space between the joint surfaces 24, 25 on the one hand and the sawing plane 21 on the other hand is successfully increased in size.

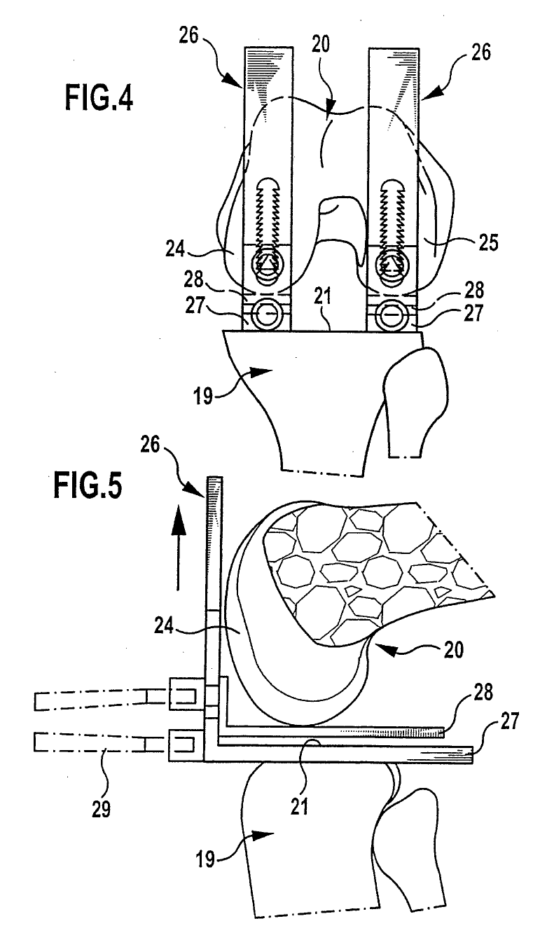

This increase in size is opposed by a resistance provided by lateral ligaments connecting the femur and the tibia; these lateral ligaments tension the distal end of the femur and the proximal end of the tibia against one another. The spreading open of the intermediate space between the femur and the tibial head takes place against the force of these ligaments, to be precise both when the knee is bent, as it is represented in FIG. 4 and FIG. 5, and when the knee is straightened. The lateral ligaments have an approximately elastic behavior at the beginning of the extension; however, the extensibility reaches a saturation value, so that a maximum extension of the lateral ligaments can in any event be reached when a force exceeding a certain value is applied, and consequently a maximum gap between the femur on the one hand and the tibial head on the other hand can be achieved.

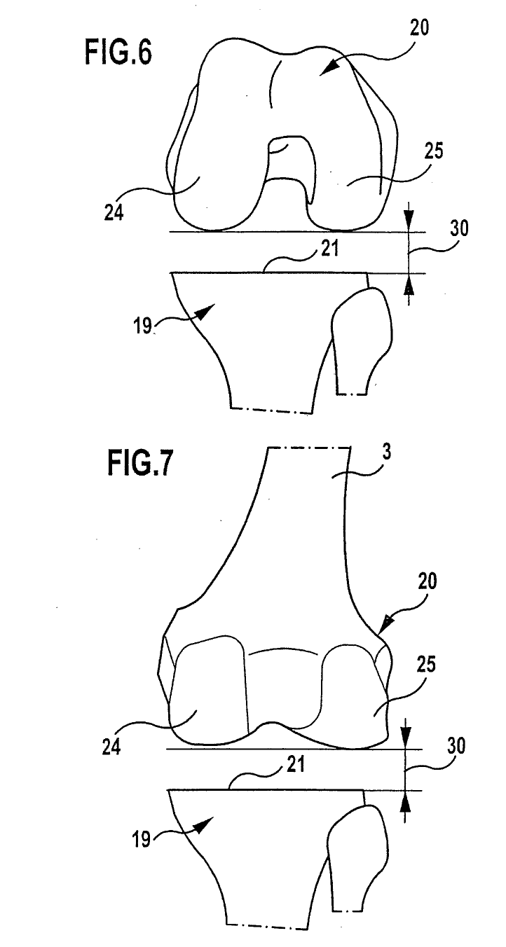

This maximum gap 30 is schematically represented in FIG. 6 in the case of a bent knee, in FIG. 7 in the case of a straightened knee. The gap width is defined by the distance of the two joint surfaces 24, 25 of the femur 3 from the sawing plane 21. In the representation of FIGS. 6 and 7, the gap width is approximately the same in the case of both joint surfaces 24, 25; however, this does not have to be the case by any means, it is quite possible for different gap widths to be produced at the two joint surfaces 24, 25 on account of malpositions or growth defects, to be precise both when the knee is bent and when the knee is straightened.

The relative position of the femur and of the tibia, which they assume both when the knee is bent and when the knee is straightened after the spreading of the distraction appliance 26, are determined by the navigation system 12, and corresponding data records are stored in the data processing system 14, therefore representing the kinematics of the knee joint to be replaced; the gap widths achieved in this way provide information on the tensioning of the two lateral ligaments.

If this investigation finds very different values for the tensioning of the lateral ligaments, the surgeon already has the possibility at this stage of changing the tensioning behavior of one of the lateral ligaments by making small incisions in it, and thereby performing a desired correction; after each correction, a distraction and determination of the relative positions of the femur and the tibial head is performed afresh, so that the result of this correction can be established immediately.

For the further procedure, geometrical data on the shape of the femur are also required. These data can be achieved with the aid of navigated engaging elements, that is engaging elements which are respectively provided with a marking element. Such an engaging element may be, for example, a contacting tip 31 with a marking element 32 (FIG. 3); with this contacting tip 31, the contours of the femur 20 can be determined point by point. Another engaging element is formed by an L profile 33 with a marking element 34. The L profile has on its inner side two planar engagement surfaces 35, 36, which are perpendicular to one another and can for example be respectively placed against the distal and dorsal ends of the femur in such a way that the engagement surface 35 engaging against the distal end is perpendicular to the previously determined mechanical axis of the femur 3 (FIG. 3). In this way, the extent of the joint surfaces 24 and 25 of the femur 3 can be determined.

From the contour data determined in this way, the distance of the joint surfaces 24 and 25 from the sawing plane 21 can be calculated for each relative position of the femur and the tibia, to be precise both medially and laterally.

Without any kind of change previously having been made to the femur, the sequences of movements which would be obtained in the knee joint if a joint endoprosthesis of a specific geometry were implanted in certain assumed positions are now simulated in a next working step. Therefore, a specific type of knee-joint endoprosthesis is initially taken as a basis, comprising for example a tibial part, an intermediate part and a femoral part; the dimensions of these parts are known, as are the relative positions of the parts of this endoprosthesis in relation to one another, which can be determined on the basis of the geometrical configuration of the joint endoprosthesis. These data are input into the data processing system in the form of a data record, for example by means of the keyboard 16.

On the assumption that the tibial part engages with a corresponding engagement surface closely against the sawing plane 21, it can be calculated in this way how corresponding engagement surfaces on the femoral part move during a movement of the joint endoprosthesis. These engagement surfaces of the femoral part correspond to sawing planes on the femur which have to be provided to adapt the femur to the femoral part. Depending on the position of the sawing planes, the femur is positioned differently in relation to the femoral part, and this of course leads to different relative positions of the femur and the tibia during the sequence of movements of the knee-joint endoprosthesis.

The position of the femoral part in relation to the femur can be varied for example by the femoral part being positioned differently in the direction of the mechanical axis of the femur (FIG. 9); it is possible that the femoral part is inclined with respect to the femur, for example about an anterior-posterior extending central axis (FIG. 10) or about a medial-lateral extending central axis (FIG. 11); the femoral part may also be displaced in the anterior-posterior direction (FIG. 12). This leads to a corresponding change to engagement surfaces of the femoral part on the correspondingly prepared femur, which is represented in FIGS. 9 to 12 by dash-dotted lines which show different positions of these engagement surfaces.

For each relative position of the femoral part in relation to the femur, the data processing system 14 can calculate the relative position of the femur and the tibia when the knee is straightened and when the knee is bent, and consequently for example also the width of the gap 30 when the knee is straightened and when the knee is bent, on the medial joint surface and on the lateral joint surface.

The surgeon consequently has the possibility of assuming different relative positions of the femoral part with respect to the femur and calculating with this assumed position the effects on the positioning of the femur and the tibia in relation to one another, and consequently also for example the widths of the gap 30 for a quite specific assumed position of the femoral part in relation to the femur.

To be able to assume such a specific position, it is advisable for example to secure a sawing template to the femur and then position the sawing template differently in relation to the femur by suitable adjusting possibilities. The plane defined by the sawing template can define for example the distal sawing surface of the femur; this defined sawing plane can be displaced or pivoted in relation to the femur by adjustment of the sawing template. If the sawing template is likewise connected to a marking element, this assumed sawing plane can consequently be spatially determined, i.e. the navigation system determines the position of this assumed sawing plane in relation to the femur.

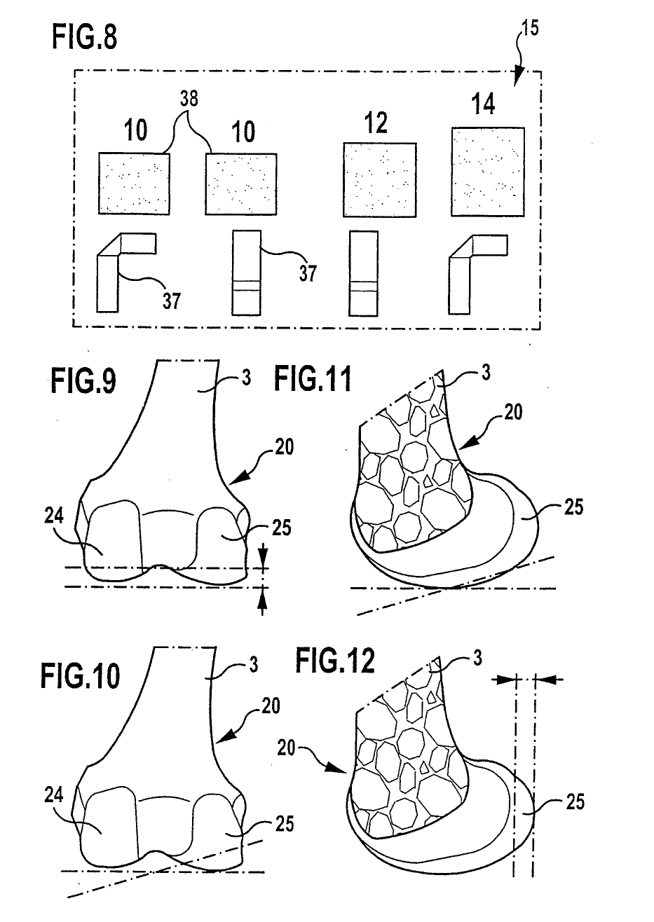

This gives the surgeon the possibility of bringing the sawing plane into a wide variety of different relative positions with respect to the femur and then calculating with this assumed position how the femur and the tibia move in relation to one another from the bent position into the straightened position on the basis of the kinematics of the endoprosthesis. For every assumed sawing plane, and consequently every assumed position of the femoral part in relation to the femur, data on the lateral and medial width of the gap 30 when the knee is straightened and when the knee is bent are thereby obtained. These data can be displayed on the display 15, as schematically shown in FIG. 8. There, the gap widths are represented in the form of rectangles next to one another, to be precise on one side for the medial joint surface and on the other side for the lateral joint surface, in both cases for a bent knee and for a straightened knee. The straight position is identified by symbols 37, the gap width by vertical bars 38, the height of which corresponds to the gap width. This height is also additionally indicated numerically.

An image of the femur in which the position of the assumed sawing plane is superposed may be additionally represented on the display 15, so that the user can immediately see how this assumed sawing plane is disposed in relation to the femur. This is of significance in particular whenever the sawing plane is inclined; the angle of inclination with respect to a surface that is perpendicular to the mechanical axis of the femur can then be read off immediately. This allows the surgeon also to check which assumed positions he should select, for example to achieve a varus-valgus correction.

Therefore, if the surgeon specifies a certain assumed position of the femoral part on the femur by adjustment of the sawing template, the widths of the gap 30 for the lateral joint surface and the medial joint surface immediately appear on the display, both for the straightened knee and for the bent knee, without any movement of the knee being necessary. The surgeon can then compare the gap widths achieved in this way with the gap widths which he wishes to achieve, whether these gap widths correspond precisely to the gap widths he previously determined on the patient's own knee with the aid of the distraction appliance 26, or whether he wishes to differ from these values in a precisely defined way, for example to make different medial and lateral gap widths similar to one another.

The surgeon can also take this opportunity to check effects on the tensioning behavior of the lateral ligaments resulting from his wish to correct a specific malposition of the knee, for example a varus-valgus malposition. He can consequently make a compromise between the positional correction on the one hand and intervention in the tensioning behavior of the lateral ligaments on the other hand, and determine the optimum position of the femoral part in relation to the femur in advance, without the femur having to be worked on in any kind of way for this purpose.

In addition, the surgeon also has the possibility of course of carrying out this method for an endoprosthesis with different dimensions or different kinematic behavior; this also results in different movements of the femur and the tibia in relation to one another. Therefore, input of the corresponding data records into the data processing system 14 additionally provides the possibility of also simulating different parts of the prosthesis and their effects on the sequence of movements, so that, on the one hand by selection of the parts of the prosthesis and on the other hand by the assumed position of the parts of the prosthesis in relation to the femur and/or the tibia, the entire sequence of movements to be expected can be simulated and compared with the desired results.

In principle, it would also be possible to carry out a corresponding calculation for different assumed positions of the tibial part in relation to the tibial head, but it is advantageous if a prescribed sawing plane 21 for a specific position of the tibial part is taken as a basis in the way described, and the variation takes place substantially by varying the prosthesis itself and/or the assumed position of the femoral part in relation to the femur.

As soon as an optimum position of the sawing plane has been found, a corresponding cut can be made on the femur with the aid of the sawing template; for example, the distal sawing surface is formed, then followed by further sawing surfaces, for example a dorsal sawing surface, which is perpendicular to the distal sawing surface. Its position is determined in a quite similar way by assuming different positions of the sawing surface and then calculating the effect of the different positioning on the kinematics. For example, the displacement of the dorsal cutting plane leads to the femoral part being positioned differently in relation to the femur in the anterior-posterior direction.

When the cuts have been made, their exact position is verified once again by placing navigated engaging elements against the cutting planes, for example the engaging element 22, so that it is ensured that the cutting plane produced also corresponds in actuality to the selected, assumed position of the sawing plane.

The method steps described are schematically summarized in the representation of FIG. 14; in particular, it can be gathered from these that the surgeon repeats the simulating process several times with different assumed positions of the femoral part in relation to the femur, until the optimum position is found.

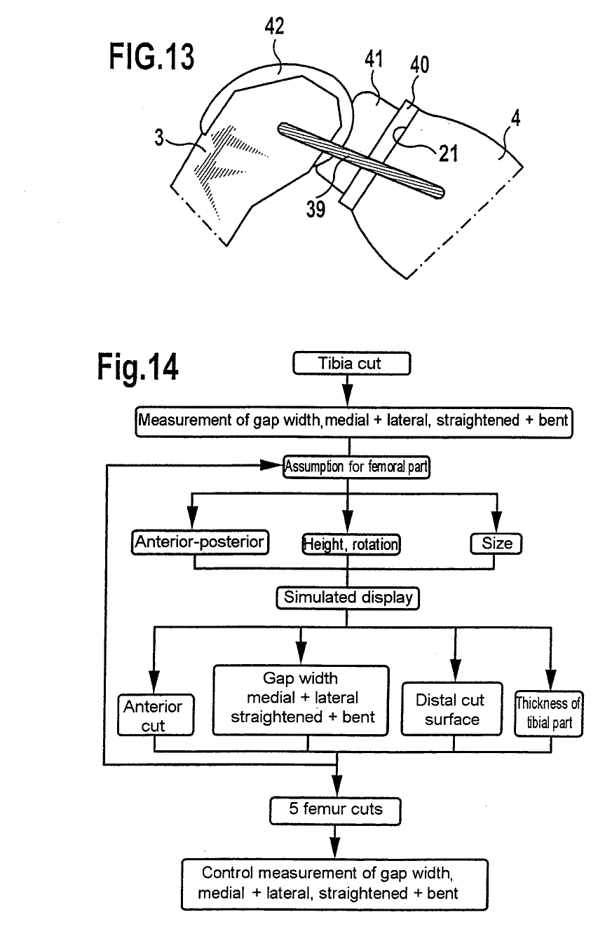

In FIG. 13 it is schematically shown how the tibial part 40, the intermediate part 41 and the femoral part 42 of the knee-joint endoprosthesis are disposed on the tibia and the femur once implantation has taken place; this representation also schematically illustrates how the tibia and the femur are tensioned with respect to one another by lateral ligaments 39.

1. Method for determining the position of the tibial part and/or the femoral part of a knee-joint endoprosthesis in relation to the proximal tibial head or to the distal femur in which the position of the femur and of the tibia are monitored by means of a navigation system, in which the distal femur and the proximal tibial head are laterally and medially displaced with a defined force into a spread position by means of a distraction appliance when the knee is straightened and bent, and the relative positions of the femur and the tibia, and consequently the size of the gap between the femur and the tibia, are thereby respectively determined, in which various virtual relative positions of the femur and the tibia are calculated according to geometrical data of the knee-joint endoprosthesis and to different assumed positions of the tibial part on the tibia and/or of the femoral part on the femur when the knee is straightened and bent, and in which an assumed position in which the virtual relative position of the femur and the tibia when the knee is straightened and bent differs from the spread position in a specified manner is determined as a selected position.

2. Method according to claim 1, wherein a selected position is determined in such a way that the virtual relative position of the femur and the tibia coincides with the spread position when the knee is straightened and bent.

3. Method according to claim 1, wherein a selected position is determined in such a way that the size of the gap laterally and medially between the femur and the tibia in the bent position and/or the straightened position of the knee is at least approximately equal.

4. Method according to claim 1, wherein, for the calculation of various virtual relative positions, the assumed position of the femoral part is displaced by displacement of the femoral part perpendicularly to the longitudinal axis of the femur while remaining parallel to itself.

5. Method according to claim 1, wherein, for the calculation of various virtual relative positions, the assumed position of the femoral part is changed by its displacement in the anterior-posterior direction.

6. Method according to claim 4, wherein, for the calculation of various virtual relative positions, the assumed position of the femoral part is changed by its displacement in the anterior-posterior direction.

7. Method according to claim 1, wherein, for the calculation of various virtual relative positions, the assumed position of the femoral part is changed by its pivoting about an anterior-posterior extending axis.

8. Method according to claim 1, wherein, for the calculation of various virtual relative positions, the assumed position of the femoral part is changed by its pivoting about a medial-lateral extending axis.

9. Method according to claim 1, wherein differently dimensioned tibial parts and/or femoral parts are taken as a basis for the calculation of various virtual relative positions.

10. Method according to claim 4, wherein differently dimensioned tibial parts and/or femoral parts are taken as a basis for the calculation of various virtual relative positions.

11. Method according to claim 5, wherein differently dimensioned tibial parts and/or femoral parts are taken as a basis for the calculation of various virtual relative positions.

12. Method according to claim 1, wherein, in the determination of the spread position, the gap between the femur and the tibial head is widened to the maximum extent.

13. Method according to claim 1, wherein, to determine the size of the gap between the femur and the tibia, the contour of the proximal tibial head and the contour of the distal femur are determined by the engagement of at least one navigated engaging element on these contours.

14. Method according to claim 13, wherein an engaging element has a contacting tip, with which various points of the contours are sensed.

15. Method according to claim 13, wherein an engaging element has a planar engagement surface, which is placed against the contour to be determined.

16. Method according to claim 15, wherein an engaging element has two mutually perpendicular engagement surfaces, which are jointly placed against the contour to be determined.

17. Method according to claim 1, wherein the size of the medial and lateral gaps in the straightened and bent positions of the knee in the respectively calculated virtual relative positions are shown on a display.

18. Method according to claim 1, wherein the position of the femoral part assumed for the determination of various virtual relative positions is displayed on a display in relation to geometrical data of the femur.

19. Method according to claim 1, wherein, before spreading open the gap between the femur and the tibial head, the proximal joint surface of the tibial head is recessed along a plane which is perpendicular to the longitudinal axis of the tibia.

20. System for determining the position of the tibial part and/or the femoral part of a knee-joint endoprosthesis in relation to the proximal tibial head or to the distal femur with a navigation system for monitoring the position of the femur and the tibia by means of marking elements which can be fixed on the femur and tibia, with a distraction appliance, which displaces the distal femur and the proximal tibial head laterally and medially with a defined force into a spread position when the knee is straightened and bent, with a data processing system, which determines the relative position of the femur and the tibia during the distraction, and consequently the size of the gap between the femur and the tibia, and calculates various virtual relative positions of the femur and the tibia according to geometrical data of the knee-joint endoprosthesis and different assumed positions of the tibial part on the tibia and/or of the femoral part on the femur when the knee is straightened and bent.

21. System according to claim 20, wherein a display which displays data corresponding to the relative position of the femur and the tibia during the distraction and data corresponding to the virtual relative positions for the purpose of their comparison is associated with the data processing system.

22. System according to claim 21, wherein the data processing system shows on the display the size of the medial and lateral gaps in the straightened and bent positions of the knee in the respectively calculated virtual relative positions.

23. System according to claim 20, wherein the data processing system displays on the display the position of the femoral part assumed for the determination of various virtual relative positions in relation to geometrical data of the femur.

24. System according to claim 20, characterized in that it comprises at least one navigated engaging element, which can be placed against the contour of the proximal tibial head and the contour of the distal femur to determine the size of the gap between the femur and the tibia.

25. System according to claim 24, wherein an engaging element has a contact tip, with which various parts of the contours are sensed.

26. System according to claim 24, wherein an engaging element has a planar engagement surface, which is placed against the contour to be determined.

27. System according to claim 26, wherein an engaging element has two mutually perpendicular engagement surfaces which are jointly placed against the contour to be determined.
